(19) United States
(12) Patent Application Publication
(10) Pub. No.: US 2008/0097595 A1
(43) Pub. Date: Apr. 24, 2008

(54) Intraventricular cardiac prosthesis

(76) Inventor: Shlomo Gabbay, New York, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/507,686

(22) Filed: Aug. 22, 2006

(51) Int. Cl. A61F 2/24

(52) U.S. Cl. 623/2.42; 604/532; 623/2.2

(57) ABSTRACT

An intraventricular apparatus includes an elongated body having a substantially tubular sidewall that extends substantially axially between spaced apart first and second ends, an opening located proximal the first end. A valve is located adjacent the first end to provide for substantially unidirectional flow of blood therethrough. At least one aperture extends through the tubular sidewall at an axial location between the valve and the second end to provide for substantially free flow of blood between an interior and an exterior of the sidewall.

### TECHNICAL FIELD

The present invention relates generally to implantable cardiac apparatuses and, more particularly, to an intraventricular apparatus that can be implanted at least partially within a patient's ventricle.

### BACKGROUND

A heart valve can become defective or damaged, such as resulting from congenital malformation, disease, or aging. When the valve becomes defective or damaged, the leaflets may not function properly. One common problem associated with a degenerating heart valve is an enlargement of the valve annulus (e.g., dilation). Other problems that may result in valve dysfunction include chordal elongation, lesions developing on one or more of the leaflets and calcification of the valve.

It is well known to utilize mechanical heart valves, such as the ball check valve, and natural tissue cardiac valves to replace defective aortic and mitral valves in human patients. One type of natural tissue heart valve typically employs a porcine valve for implantation in a human, as they are very similar to human valves of appropriate size and generally are easy to procure. Typically, the porcine valve is fixed by chemically treating it, such as with an appropriate glutaraldehyde solution. The treated porcine valve further may be mounted into a stent to support the valve at a fixed position.

In order to surgically implant a heart valve into a patient, the patient typically is placed on cardiopulmonary bypass during a complicated, but common, open-chest and open-heart procedure. In certain situations, an individual requiring a heart valve replacement may be sufficiently ill, such that placing the individual on cardiopulmonary bypass may pose too great of risk. As one example, such individuals may correspond to a class of patients who may have severe aortic valve insufficiency. Patients with aortic valve defects often may also exhibit calcification of the aortic valve and the aorta. When the aorta is calcified, there are increased risks associated with performing cardio pulmonary bypass, as is typically performed for aortic valve replacement procedures. Additionally, patients having a diseased or defective aortic valve may be too ill to survive conventional open-heart surgery, which may include cardio pulmonary bypass.

### SUMMARY

The present invention relates to an intraventricular apparatus that can be implanted at least partially within a patient's ventricle. For example, one embodiment can be utilized to replace the function of a patient's aortic valve and another embodiment can be utilized to replace the function of a patient's mitral valve.

One aspect of the present invention provides an intraventricular apparatus that includes an elongated body having a substantially tubular sidewall that extends substantially axially between spaced apart first and second ends, an opening located proximal the first end. A valve is located adjacent the first end to provide for substantially unidirectional flow of blood therethrough. At least one aperture extends through the tubular sidewall at an axial location between the valve and the second end to provide for substantially free flow of blood between an interior and an exterior of the sidewall.

Another aspect of the present invention provides an intraventricular apparatus that includes an elongated substantially cylindrical sidewall that extends substantially longitudinally between spaced apart first and second ends. An opening at the first end permits flow of fluid relative to an interior lumen of the elongated body. A valve is located between the opening and an axial length of the sidewall that extends between the valve and the second end. The valve permits substantially unidirectional flow of fluid through the valve. A plurality of apertures through an inflow section of the sidewall between the valve and the second end to permit substantially free flow of blood between an interior of the inflow section and a location external to the inflow section. The apparatus also includes at least one support member to maintain a substantially cylindrical configuration of the sidewall near at least some of the plurality of apertures.

Still another aspect of the present invention provides an intraventricular apparatus comprising that includes elongated means for providing a lumen that extends within a substantially cylindrical sidewall between spaced apart first and second ends. The apparatus also includes means for providing an opening at the first end of the elongated means that permits flow of fluid through the opening relative to an interior of the elongated means. The apparatus also includes means for providing substantially unidirectional flow of fluid axially through a portion of the elongated means proximal the first end of the elongated means. The apparatus also includes means for permitting substantially free flow of the fluid through the sidewall of the elongated means. The apparatus also includes means for maintaining a substantially cylindrical configuration of the sidewall of the elongated means between the second end and the means for providing unidirectional flow of fluid.

### BRIEF DESCRIPTION OF THE DRAWINGS

FIG. 1 depicts an example of an intraventricular cardiac apparatus according to one embodiment of the present invention.

FIG. 2 depicts an example of an intraventricular cardiac apparatus according to another embodiment of the present invention.

FIG. 3 depicts part of the apparatus of FIG. 2 illustrated in a second condition, as can be part of an implantation procedure implemented, according to aspect of the present invention.

FIG. 4 depicts a cross-sectional view of the apparatus of FIG. 2 taken along line 4-4.

FIG. 5 depicts an example of an intraventricular cardiac apparatus according to yet another embodiment of the present invention.

FIG. 6 depicts a cross-sectional view of the apparatus of FIG. 5 taken along line 6-6.

FIG. 7 depicts part of an implantation procedure that can be used for implanting an intraventricular cardiac apparatus according to an aspect of the present invention.

FIG. 8 depicts another part of an implantation procedure that can be used for implanting an intraventricular cardiac apparatus according to an aspect of the present invention.

FIG. 9 depicts another part of an implantation procedure that can be used for implanting an intraventricular cardiac apparatus according to an aspect of the present invention.

FIG. 10 depicts part of an implantation procedure that can be used for implanting an intraventricular cardiac apparatus according to an aspect of the present invention.

FIG. 11 depicts part of a procedure that can be used for ensuring proper placement of an intraventricular cardiac apparatus being implanted according to an aspect of the present invention.

FIG. 12 depicts an intraventricular cardiac apparatus implanted according to an aspect of the present invention.

FIG. 13 depicts part of a procedure, employing an intraventricular apparatus, which can be implemented according to an aspect of the present invention.

FIG. 14 depicts an example of an intraventricular cardiac apparatus according to another embodiment of the present invention.

FIG. 15A depicts a cross-sectional view of the apparatus of FIG. 14 taken along line 15A-15A.

FIG. 15B depicts a cross-sectional view of the apparatus of FIG. 14 taken along line 15B-15B.

FIG. 16 depicts the intraventricular cardiac apparatus of FIG. 14 implanted according to an aspect of the present invention.

FIG. 17 depicts an example of an intraventricular cardiac apparatus according to yet another embodiment of the present invention.

FIG. 17A depicts a cross-sectional view of the apparatus of FIG. 17 taken along line 17A-17A.

FIG. 18 depicts a partial cross-sectional view of the apparatus of FIG. 17 located within a tubular enclosure and having a reduced cross-sectional configuration.

FIG. 19 depicts the apparatus of FIG. 17 and 18 be implanted by ejecting the apparatus from the enclosure shown in FIG. 18.

FIG. 20 depicts the intraventricular cardiac apparatus of FIG. 17 implanted according to an aspect of the present invention.

### DETAILED DESCRIPTION

The present invention relates generally to an intraventricular apparatus and to methods of using the apparatus.

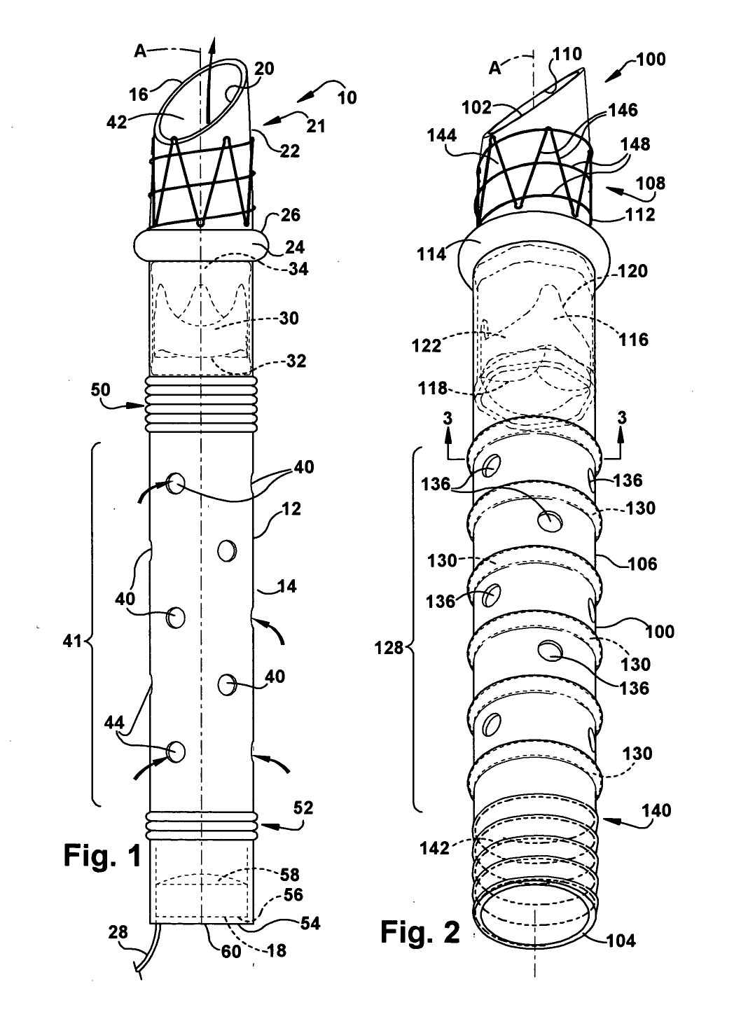

FIG. 1 depicts an example of an intraventricular apparatus 10 that can be implemented according to an aspect of the present invention. The apparatus 10 includes an elongated body 12 having a substantial tubular sidewall 14 extending between spaced apart first and second ends 16 and 18, respectively. An outflow opening 20 is located proximal to first end 16. The outflow opening 20 can be formed through the sidewall of the sidewall 12 or it may be located at the end 16. The outflow opening 20 can be defined by a perimeter edge that is oriented transversely relative to a central axis A that extends through the apparatus 10. For example, the perimeter edge can line in a plane that is at a predetermined angle (e.g., about 45 degrees) relative to the central axis A. Alternatively, the perimeter edge of the opening 20 can be oriented about 90 degrees or at other angles relative to the central axis A.

The opening 20 also has a diameter sufficient to permit the flow of blood through the opening and, when implanted, into a patient's aorta. It will be appreciated that the size of the opening 20 can be designed to provide desired hemodynamic characteristics. As an example, an internal diameter of about 15 mm for the opening 20 should provide hemodynamic characteristics comparable to a 21 mm diameter prosthetic heart valve (e.g., a size 21 mm valve).

The outflow portion of the apparatus proximal the end 16 can define a cannula tip 21 that is dimensioned and configured for insertion through an aortic valve annulus and into a patient's aorta. The cannula tip 21 thus includes a tubular sidewall portion 22 that extends from the first end 1b and terminates at an axially spaced apart location at an intermediate portion of the body 12. As depicted in the example of FIG. 1, the sidewall portion 22 of the cannula tip 21 can have a generally frusto-conical configuration that has a smaller cross sectional diameter at the end 16 and increases to a larger diameter at a location spaced axially apart from the end 16 proximal the body 12 portion of the apparatus 10. The cannula tip 22 proximal the first end 16 can include a substantially flexible substrate material, such as plastic, metal or other material capable of maintaining a desired configuration to provide for the flow of fluid therethrough. The interior and exterior surfaces of the cannula tip can be covered with biocompatible material, such as a biological material.

A bulbous portion or protruding member 24 extends radially outwardly from the sidewall 14 near the juncture between the cannula tip 21 and the intermediate part of the body 12. The protruding member 24 can be formed of a flexible material (e.g., a natural or synthetic material or a combination of natural or synthetic materials) that circumscribes the base of the tip portion 21 and defines an outflow shoulder portion 26.

The protruding member 24 can be inflatable, such as by including an inflatable member. For example, one or more balloons that circumscribe the tubular sidewall can be selectively inflated or deflated by controlling the amount of a fluid within an interior volume of the protruding member. The amount of fluid within the interior volume defines the radial distance that the protruding member extends outwardly relative to the tubular sidewall 14. The protruding member 24 can be inflated through an internal lumen or conduit 28 that extends axially from the protruding member to being accessible at the second end 18. A suitable source of inflation fluid, such as a syringe or pump, can be utilized to inject corresponding fluid into the protruding member to cause corresponding inflation or to remove fluid for deflation thereof. As an example, the corresponding lumen or conduit 28 can be sandwiched between inner and outer sidewall portions of the tubular body 14 or a separate thin conduit can extend axially through an interior or exterior of the apparatus 10 to provide for fluid communication to an interior volume of the inflatable protruding member 24. The amount of fluid within the interior volume defines the radial distance that the protruding member extends outwardly relative to the tubular sidewall 14. Alternatively, the diameter and size of the protruding member 24 can be substantially fixed relative to the tubular sidewall 14 from which it extends.

The apparatus 10 can also include a valve, schematically illustrated at 30, which is located axially spaced apart form the first end 16. For example, the valve 30 can be located axially adjacent the cannula tip 21 within a length of the tubular sidewall 14 that extends to the second end 18. The protruding member, thus, can be located axially near or at the juncture between the cannula tip 21 and an outflow end of the valve. The valve 30 has an inflow end 32 and an outflow end 34 with corresponding structure located between the inflow and outflow ends to provide for substantially unidirectional flow of blood through the valve. The valve 30 defines means providing for a substantially unidirectional flow of blood therethrough, such as in a direction from the second end 18 through the valve and toward the opening 20. It is to be understood that the valve can be implemented as a natural tissue valve, a mechanical valve or a biomechanical valve (e.g., that includes mechanical and natural tissue components). As but one example, the valve 30 can be implemented as an expandable type of heart valve prosthesis, such as shown and described in co-pending patent application Ser. No. 10/266,380, which was filed Oct. 8, 2002, and which is incorporated herein by reference. As an example, the expandable type of heart valve prosthesis can be inserted with a tubular section of the sidewall proximal the tip portion 21, then and expanded to a desired cross-sectional dimension that affords desired coaptation of the valve 30. One or more sutures can be applied to fix the axial and radial position of the valve relative to the sidewall 14. Alternatively, the heart valve can be attached near the tip portion 21 by other means that may vary depending on the type of valve being used.

One or more apertures 40 extend through the tubular sidewall 14 at respective spaced apart locations to define an inflow section 41 of the apparatus 10 between the valve 30 and the second end 18. The apertures 40 correspond to inflow openings into which blood can enter the apparatus for flow axially through the valve 30 and out the outflow opening 20. The size of the apertures 40 can vary according to the diameter of the tubular portion 12. As one example, the aggregate area of the apertures 40 can at least approximate the cross-sectional area of the outflow end of the valve (e.g., about 5 cm² total area for a 2.5 cm diameter valve). The plurality of apertures 40 can be formed through the sidewall 14 at circumferentially and axially spaced apart locations the tubular sidewall portion 14.

The apparatus 10 can include inner and outer layers of a biocompatible flexible material. For example, an interior surface 42 and an outer surface 44 of various parts of the apparatus 10 can be covered with (or be formed from) the biocompatible flexible material. The flexible material can be utilized in the form of one or more sheets or tubes that are attached to the apparatus 10 to cover the respective surfaces 42 and 44. In the example of FIG. 1, the portion of the tubular sidewall 14 through which the apertures extend can be formed of one or more layers of such material, such as in the form of tube of such flexible material. The flexible material can be a biological material (e.g., natural tissue material, such as animal pericardium, collagen, dura matter or the like). Alternatively, the flexible material can be synthetic material or a combination of biological and synthetic materials.

As one example, the biocompatible material can be formed from one or more sheets of a NO-REACT® tissue product, which are commercially available from Shelhigh, Inc., of Millburn, N.J. The NO-REACT® tissue products help improve the biocompatibility of the apparatus 10, thereby mitigating the likelihood of a patient rejecting an implanted prosthesis that includes the apparatus. The NO-REACT® tissue also has been shown to resist calcification as well as promote formation of endothelial cells when implanted in vivo.

The apparatus 10 can also include one or more axially extending tubular portions 50, 52 located between the valve 30 and the end 18 of the tubular sidewall 14. The tubular portions 50 and 52 are more pliant than the tip portion 22. The tubular portions 50 and 52 can be configured to permit axial elongation and/or compression of the apparatus, radial deflection of the apparatus relative to the axis A, as well as accommodate torsional stress that may be applied to the apparatus when implanted. As one example, the tubular portions 50 and 52 can be formed of a corrugated length of a tubular material that permits an axial movement, including axial compression and axial extension of the portion 50. Additional structural memory can be provided by employing one or more helical springs of an elastic material mounted to or within the corrugated tubular material. The corrugated portion 50 may also permit a flexion of the sidewall that is transverse to the axis A or rotational or torsional stress that may be applied.

In this way, when the apparatus 10 is implanted within a patient's heart, such that the ends 16 and 18 are substantially fixed relative to the patient's heart, as the heart beats and moves accordingly, the apparatus 10 can conform to such movement due to the ability to stretch, compress and flex at the tubular portions 50 and 52. Those skilled in the art will understand and appreciate various types of flexible biocompatible materials and structures that can be utilized to afford axial and radial movement at the respective tubular portions 50 and 52. While the portions 50 and 52 are illustrated as being corrugated other types of configurations to permit such movement can also be utilized.

The apparatus 10 can also include one or more support elements dimensioned and configured to help maintain a substantially cylindrical configuration of the tubular sidewall 14. For instance, such support elements can be placed adjacent the respective apertures. For example, the support elements can be implemented as one or more elastic rings positioned axially between the respective apertures (e.g., on axially opposed of some or each of the respective apertures) stored to maintain a desired cylindrical circular configuration of the sidewall portion 14. Alternatively, an elongated helical spring or springs or other resilient structures can be utilized to provide for a desired configuration. The apertures 40 can be formed in spaces between axially adjacent rungs of the spring or between axially spaced apart support structures. The support element or elements can be positioned radially between respective layers (e.g., formed as substantially concentric tubes) of the biocompatible flexible material that forms the inflow section 41 of the tubular sidewall 14.

The apparatus 10 can also include a plug 54 that can be mounted at an opening 56 located at the second end 18. The plug 54 can be formed of a substantially pliant material, such as silicone or a rubber-like material, configured to fit matingly within the opening 56 at the second 18. Alternatively, other types of fittings, threaded members and the like can be utilized as a plug for closing the opening 56 at the second end 18. The plug 54 can also include a convex distal end 58 that is spaced apart from a proximal end 60 of the plug. The axial length of the plug can be less than the distance from the end 18 to the closest aperture 40. Other structures can be used to close or plug the second end 18, such as including by forming the apparatus 10 with a permanently closed second end.

FIGS. 2 through 4 depict an example of another intravascular apparatus 100 that can be implemented according to the aspect of the present invention. The apparatus 100 has a substantially tubular configuration that extends between respective ends 102 and 104 that are spaced apart by an associated sidewall 106. The apparatus 100 includes a tip portion 108 located at the first end 102. The tip portion 108 can define a cannula tip similar to as shown and described with respect to FIG. 1. Generally, the cannula tip portion 108 includes an opening 110 at the end 102, such as may be at an angle (e.g., about 45 degrees relative to the central axis A extending through the apparatus. The cannula tip portion 108 thus extends from a proximal end 112 and terminates in the end 102. The sidewall of the cannula tip 108 extends between the proximal end 112 and the distal end 102 can be formed of a substantially flexible material. The cannula tip 108 can also be covered with a biocompatible material that covers a substrate having the desired configuration and rigidity the interior and exterior surfaces thereof. For example, as discussed with respect to FIG. 1, an animal tissue material having been treated and substantially detoxified (e.g., a NO-REACT tissue product) can be utilized to provide the covering for the cannula tip portion 108 as well as covering the other surfaces of the apparatus 100.

The apparatus 100 also includes a radially protruding portion 114 that can be similar to that shown and described with respect to FIG. 1. For instance, the protruding member can circumscribe the sidewall 106 near the proximal end 112 of the cannula tip 108.

A valve 116 is located within the sidewall 106 proximal the protruding portion 114. The valve 116 can be a natural tissue heart valve prosthesis, such as may be shown and described in the above-incorporated patent application. The valve 116 can include an inflow end 118 spaced axially apart from an outflow end 120, and further can include a support structure 122 that helps maintain the valve in a desired configuration to provide for proper coaptation of the valve leaflets to provide for substantially unidirectional flow of blood through the valve. While the valve 116 is shown and described as a natural tissue valve prosthesis, those skilled in the art will understand and appreciate that other types of valves can also be utilized in the apparatus 100. For instance, other types of configurations of natural tissue valve prosthesis as well as mechanical valves or biomechanical valves can also be utilized.

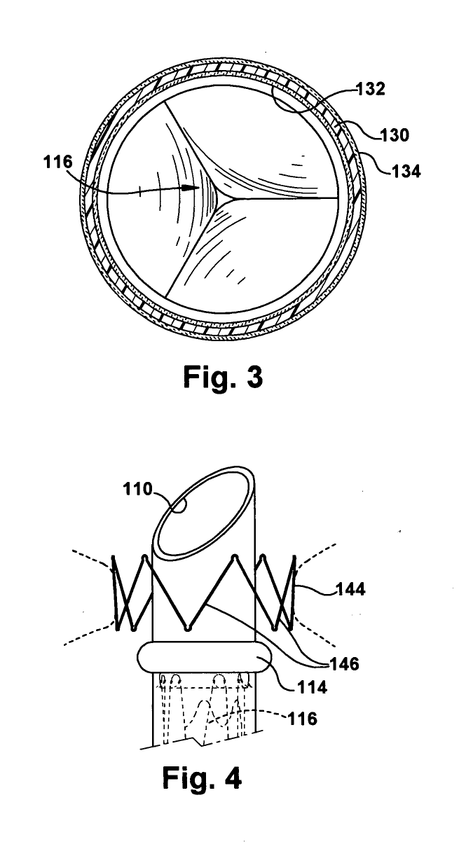

The portion of the sidewall 106 extending between the valve 116 and the end 104, which defines an inflow section 128, can be supported by a plurality of support members 130. In the example of FIGS. 2 through 4, the support members 130 are illustrated as annular structures that are spaced axially apart from each other along the length of the inflow section 128 (e.g., short cylindrical rings). As thus shown in FIG. 3, the support members 130 can be mounted between corresponding tubular sheets 132 and 134 of a biocompatible flexible material, such as described herein. A plurality of apertures 136 can be formed through the sidewall, including through both of the sheets 132 and 134. The apertures 136 can be at axial spaced apart locations between adjacent pairs of the respective support members 130. In this way, the softer more compliant sheets of flexible material that extend between the apertures can be supported by the respective rings to maintain a desired configuration of the inflow section 128 of the sidewall 106.

A proximal elongated portion 140 of the tubular sidewall 106 between the inflow section 128 and the proximal end 104 can be configured to permit axial elongation, compression and radial deflection relative to the central axis A. In this regard, the portion 140 can include one or springs (e.g., helical springs) 142 encapsulated within a biocompatible material. For instance, the springs 142 can be mounted between the sidewall sheets 132 and 134 of the biocompatible material. The same biocompatible materials further may form the interior and exterior surfaces of the other parts of the sidewall 106. Additionally, to facilitate elongation compression and transverse movement of the portion 140 relative to the axis A, the sidewall (that is formed by the interior and exterior sheets 132 and 134 of biocompatible materials) may be corrugated or otherwise configured similar to a bellow or an accordion to permit desired movement thereof. Those skilled in the art will understand and appreciate various types and configurations of materials that can provide for suitable movement and sufficient support at the proximal end portion 140.

At the distal end portion 108, an additional support structure 144 may be utilized. The support structure 144 in the examples of FIGS. 2 and 4 is depicted as an expandable type stent structure having a plurality of axially extending support members 146 that extend between axial junctures configured to expand the support structure from a compressed state (shown in FIG. 2) to its expanded state in FIG. 4. The support structure 144 can be self-expanding or otherwise be expandable by mechanical or other means. In the example of FIG. 2, the support structure 144 is compressed against the exterior sidewall of the cannula tip member 108 and held in such positions. For example, one or more sutures (or other retaining structure 148) can be applied around the exterior of the support 144 structure to hold it against the exterior sidewall of the cannula tip portion 108. Alternatively, a tubular sheet of a corresponding biocompatible material can be applied around the structure to hold in its desired compressed orientation. In its compressed condition, insertion of the apparatus 100 into and through a patient's heart valve can be facilitated. Once in an appropriate position, the retaining structure (e.g., suture and/or tube of tissue) 148 can be removed and the support structure 144 can be expanded into engagement with surrounding tissue. The support structure 144 can also include spikes that extend into the surrounding tissue to help maintain its relative axial and rotational positions. In this way, the opening 102 can remain unobstructed to facilitate the flow of blood from the opening and into a patient's aorta.

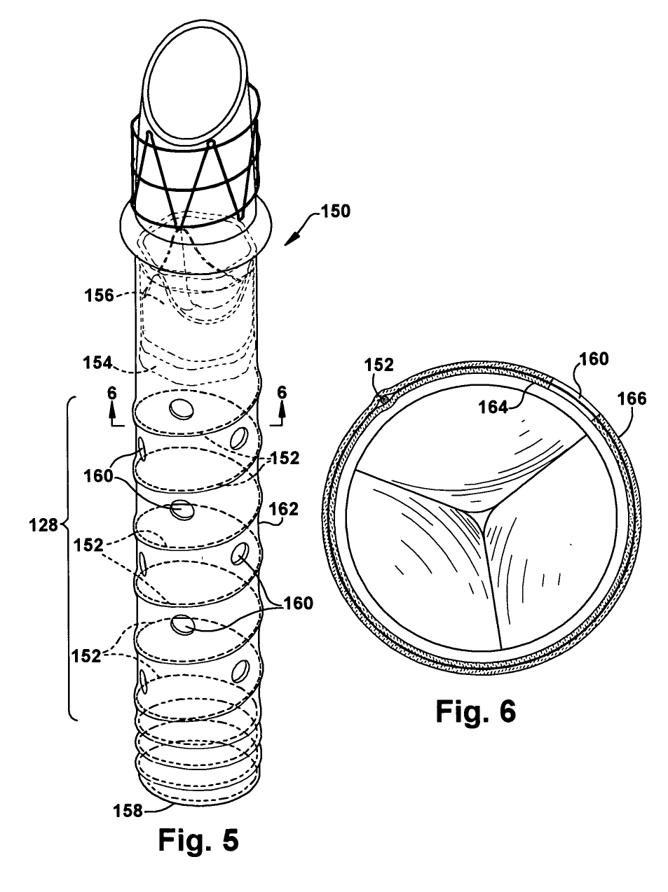

FIGS. 5 and 6 depict an alternative embodiment of an intraventricular apparatus 150 that can be implemented according to an aspect of the present invention. The apparatus 150 is substantially similar in many respects to the apparatus 100 shown and described with respect to FIGS. 2 through 4. However, the supporting structure that is utilized to maintain interior lumen of the tubular portion in a desired configuration is implemented as one or more springs (or windings) 152 that extend axially from an inflow end 154 of the valve 156 to a proximal end 158 of the apparatus 150. The axial spacing between adjacent windings 152 at a proximal end portion 159 can be smaller than the axial spacing at an inflow portion 163 of the apparatus 150 through which a plurality of apertures 160 extend through the sidewall. The plurality of apertures 160 thus are formed through an inflow section 163 of the sidewall 162 between adjacent and axially spaced apart coils of the spring 152.

While the spring 152 in the example of FIG. 5 is depicted as a generally helical configuration, those skilled in the art will understand and appreciate that other types of spring support structures can also be utilized. For example, a spring support formed of a plurality of axially extending support members (similar to the support structures as applied over the cannula tip portion of the apparatus) can also be used. As shown in FIG. 6, the spring 152 can be encapsulated or covered by a pair of concentric tubular sheets 164 and 166 of a flexible biocompatible material. The sheets 164 and 166 can be used to cover the interior and exterior surfaces of the apparatus 150.

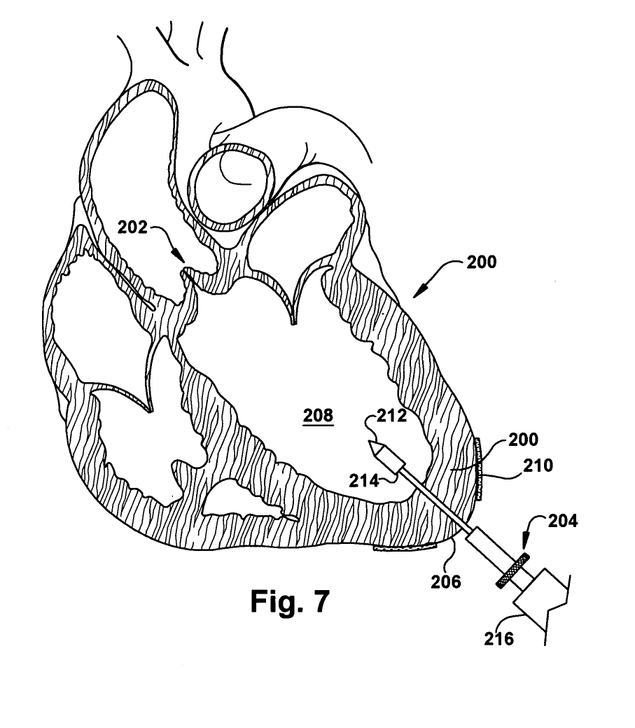

FIGS. 7 through 12 depict different parts of a procedure that can be utilized to implant an intraventricular apparatus according to an aspect of the present invention. Those skilled in the understand and appreciate that various embodiments, including those shown and described herein, can be implanted in the manner set forth in FIGS. 7 through 12. Those skilled in the art will understand and appreciate that various other embodiments of the apparatus can also be implanted in a similar way. Advantageously, the procedure being described can be performed in the absence of cardiopulmonary bypass. As a result, the device can be utilized for a more expansive range of patients.

Referring to FIG. 7, the procedure begins by cutting a hole through the heart 200 muscle to provide a path from a location external to the heart to the aortic valve 202. The aortic valve 202 can be stenotic and calcified. As an example, a myocardial punch apparatus 204 can be inserted through the apex 206 of the heart 200 and into the patient's left ventricle 208. Prior to inserting the myocardial punch apparatus 204 through the heart muscle 200, a sheet 210 of a biocompatible material can be placed in position over the apex 206 of the patient's heart 200. For example, the sheet 210 can be in the form of an annular sheet of an animal tissue material (e.g., pericardium) and can be attached the exterior of the patient's heart so that an inner peripheral edge of the sheet 210 circumscribes (or surrounds) the portion of the apex 206 of the heart through which the hole is to be formed.

The cutting apparatus 204 can include a substantially sharp and pointed tip 212 at a distal end thereof the facilitate insertion through the heart muscle 200. A substantially circular cutting edge 214 is spaced at a proximal side of the tip portion 212. The sharpened cutting edge 214 can be activated and retracted axially towards the proximal end 216 of the cutting apparatus 208 located external to the heart 200. The axial retraction of the cutting edge 214 cuts a corresponding hole 218 in the heart muscle 200 dimensioned according to the diameter of the cutting edge. The portion of the heart muscle 200 at the apex 206 can remain within an interior chamber of the cutting tip 212 to facilitate its removal from the heart. Those skilled in the art will understand and appreciate that different size cutting apparatuses can be utilized to form the corresponding hole at the apex of the patient's heart to provide a path from the exterior of the heart through the myocardium and into the ventricle 208. As the cutting apparatus 204 (and plug of heart muscle) is retracted from the heart 200, a finger or other instrument can be utilized to occlude the wound (corresponding to the hole) 218 and thereby mitigate blood loss during the procedure.

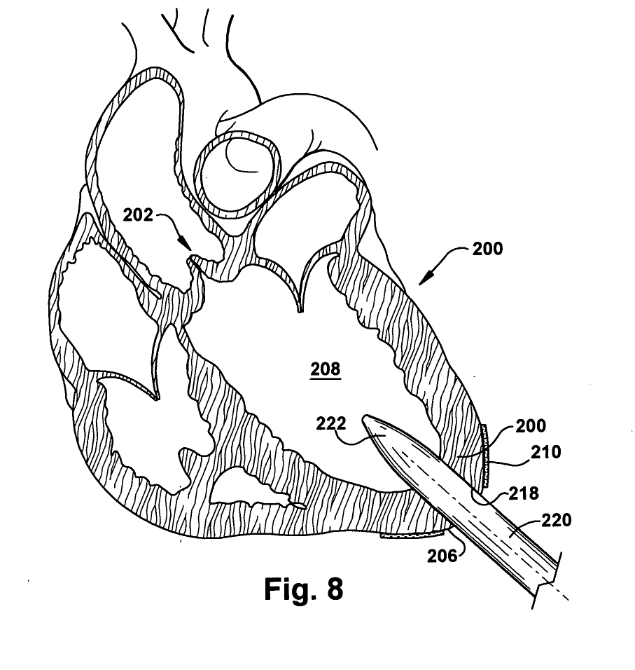

In FIG. 8, the initial hole 218 created by the cutting apparatus 204 can be dilated by a trocar or other apparatus 220 having a diameter that is greater than the size of the plug removed from the heart muscle 200. For example, a trocar 220 can include an elongated body having a tapered end 222 that is inserted through the hole 218 created by the cutting apparatus 204 and rotated to facilitated insertion into the hole. A series of different trocars of varying increasing sizes can be utilized until a sufficiently sized diameter has been formed at the apex 206 of the patient's,heart.

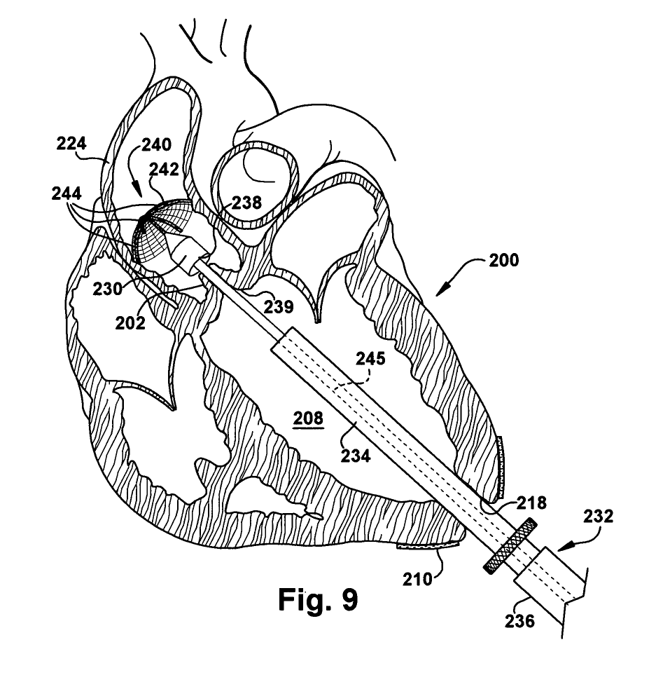

In certain circumstances, a patient's aortic valve may be significantly calcified or otherwise damaged such that it may be desirable to remove at least a significant portion of the valve prior to implanting the apparatus into the heart. Accordingly, FIG. 9 illustrates an approach that can be utilized to remove portions of the aortic valve and create an appropriate opening through which a distal end portion of the intraventricular apparatus can be inserted. The aortic valve can be the patient's native valve or a previously implanted prosthetic valve. Portions of the aortic valve may be removed, for example, as the valve becomes stenotic or occluded or if the valve is otherwise sufficiently calcified to warrant its removal prior to implantation of the apparatus.

In the example of FIG. 9, another cutting apparatus 232, which may be the same or different from that utilized in the creation of the hole at the apex 206 of the patient's heart (FIG. 7), can be employed to excise portions of the patient's aortic valve 202. As shown in FIG. 9, a cutting tip 234 of a cutting apparatus 232 is inserted through the hole 218 at the apex 206 and through the patient's aortic valve 202 and into the patient's aorta 224. The cutting apparatus 232 includes an elongated section 234 that extends between a proximal body portion 236 and the cutting tip 230. The cutting tip 230 is inserted through the aortic valve 202 so that a corresponding cutting surface 238 is positioned at least partially through the valve 202 so as to engage the aortic valve and thereby removably cut the portions engaged thereby as the cutting tip is retracted axially toward the body portion 236. The removed portion of the valve 202 can be retained in the tip portion 232. For example, a corresponding trigger or other activation component can be activated to cut through the aortic valve 202 such that the cutting surfaces of the punch are brought together to cut and remove the leaflet tissue contained therein. The cutting apparatus 232 can then be withdrawn from the heart 200 such that the removed tissue remains within the cutting tip 230.

A trap or collector 240 can be utilized to collect debris that might be dislodged during the cutting of the aortic valve. For example, the trap 240 can include a mesh screen 242 that permits substantially free flow of blood through the screen, but prevents debris from flowing through the screen. As one example embodiment, the screen 242 can be configured similar to an umbrella having a web of a mesh material that covers a plurality of radially extending supports 244. Like an umbrella, the supports 244 can be moved axially distally to open the screen 242, such as to contact a peripheral sidewall of the patient's aorta. In the example of FIG. 9, the trap is configured to traverse an elongated lumen 245 extending through the cutting apparatus 232, such that the trap 240 can be moved into position, opened and closed through the cutting apparatus. After the screen 242 has been opened and into engagement with the interior wall of the aorta, the cutting apparatus 232 can be activated to excise the aortic valve. Debris will be caught in the mesh screen. After cutting is completed, the mesh screen 242 can be closed, such as by causing the radially extending supports to move proximally. In the closed position, the trap 240 can be withdrawn from the patient's heart 200, either through the lumen 245 of the cutting apparatus 232 or concurrently with the withdrawal of the cutting apparatus.

It is to be understood that different sized cutting apparatuses can be utilized to create a larger opening through the aortic valve by cutting with increasingly larger diameter tip portions. This can be done over a series of cutting steps similar to that described above. Those skilled in the art will understand and appreciate other approaches and different tools that can be utilized to remove portions of the patient's aortic valve to provide space for implanting the tip portion of the intraventricular apparatus.

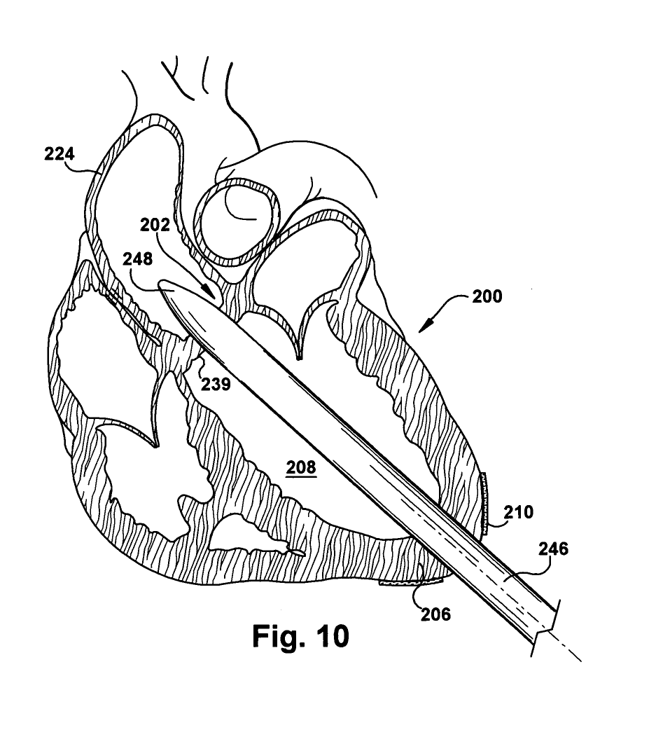

FIG. 10 depicts an example of which an elongated trocar apparatus 246 having a tapered tip portion 248 is inserted through the hole at the apex 206 of the patient's heart and through the aortic annulus where the aortic valve tissue has just been removed. The trocar 246 thus can be employed to dilate the aortic annulus (similar to as was done at the apex of the patient's heart 200) to facilitate insertion of the intraventricular apparatus.

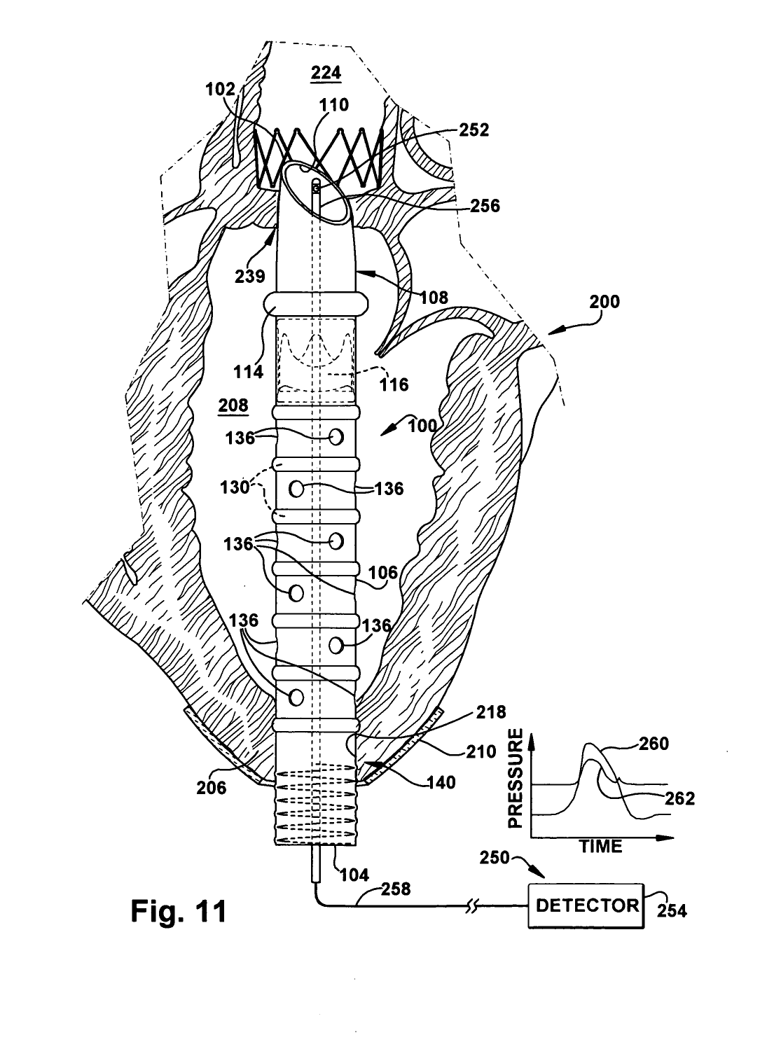

FIG. 11 depicts an intraventricular apparatus being inserted from the exterior of the heart through the hole 218 at the apex 206 through the ventricle 208 and into the aorta 224. Reference numbers shown and described with respect to the apparatus 100 in FIG. 11 are the same as shown and introduced with respect to the example embodiment shown and described with respect to FIG. 2. It is to be understood and appreciated, however, that other embodiments of intraventricular apparatuses (e.g., including FIGS. 1 and 5 or otherwise within the scope of the appended claims) can be implanted and utilized in a similar manner without departing from the spirit and scope of the present invention.

Thus, in FIG. 11, the cannula tip portion 108 of the apparatus 100 is being inserted through the aortic annulus 239 and into the interior of the arch of the aorta 224. The apparatus 100 can be inserted into the heart 200 until the protruding member 114 engages the aortic annulus 239. The protruding member 114 may be expanded to a larger cross section to provide a sealing engagement and contact between the apparatus 100 and the aortic annulus 239. The proximal end 104 of the apparatus 100 should approximate the apex 206 of the patient's heart 200 so that the elongated apparatus extends from the apex through the ventricle 208 and partially into the aortic arch. Excess length of the proximal portion 104 of the apparatus 100 can be removed (e.g., by cutting) so that the proximal end does not extend from the heart when the protruding member 114 engages the annulus 239.

Since the apparatus 100 can be implanted in the absence of cardiopulmonary bypass on a beating heart, a pressure detection system 250 can be employed to facilitate proper placement of the cannula tip portion 108 into the aorta 224. For example, a pressure transducer or sensor 252 can be positioned adjacent the distal end 102 of the apparatus. The pressure transducer or sensor 252 can be communicatively coupled to a detector 254 that is located remotely external to the heart 200. The pressure transducer or sensor 252 provides a corresponding signal indicative of the pressure. The pressure signal can vary to reflect corresponding diastolic and systolic pressure to which the pressure transducer or sensor 252 is exposed. As an example, the pressure transducer or sensor 252 can be mounted at a distal end of an elongated rod (e.g., a catheter) 256 that extends axially through at least part of the apparatus 100, such as including through the valve 116. The elongated rod 256 can be coupled to the detector via a connection 258. The transducer or sensor 252 thus provides an electrical signal to the detector 254 through the connection 258 indicative of the sensed pressure, based on which the detector provides a corresponding indication of the pressure (e.g., visual and/or audible indication of pressure).

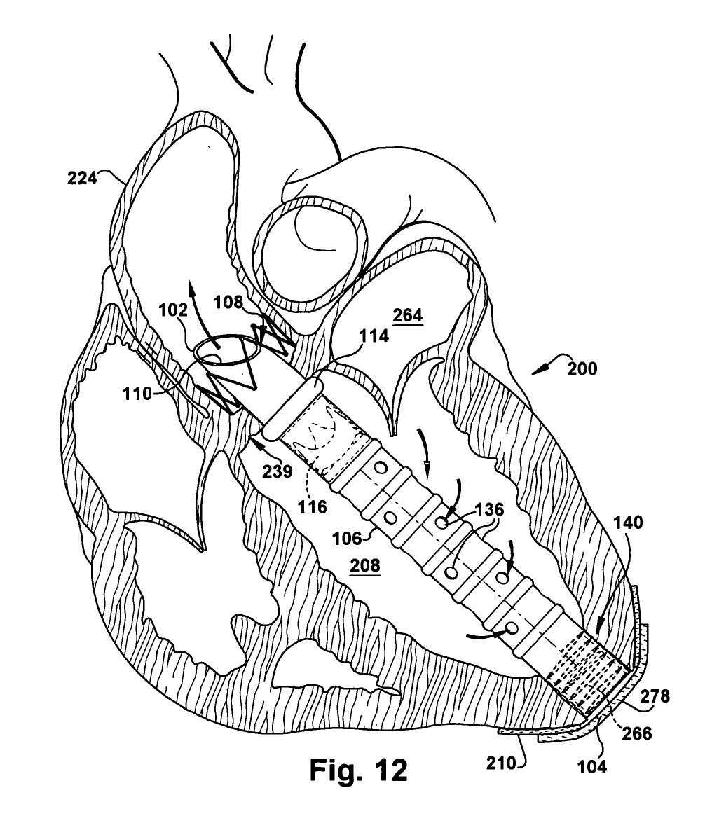

By discerning the pressure (or a detected change in the sensed pressure or a change in the associated pressure curve over time), the surgeon can determine when the cannula tip has been inserted into the aorta beyond the aortic annulus 239. For instance, as the apparatus 100 is inserted into the aorta 224, the pressure being detected as a function of time will change from corresponding to a ventricular pressure curve 260 to an aortic pressure curve 262 commensurate with the insertion of the tip 108 through the aortic annulus 239. When that the protruding member 114 engages the patient's aortic annulus, thereby mitigating peri-valvular leakage, the distinction may be more noticeable indicating that the apparatus is at the proper implantation position. An additional flange (not shown) can be provided to extend radially outwardly from the tip portion at a location between the end 102 and the protruding member 114 to further mitigate peri-valvular leakage and on the aortic side of the annulus 239. While implantation may occur based on the pressure only, it will be understood that other equipment, such as 2-D echo, X-ray or other like devices can be utilized separately or in conjunction with the pressure detection system 250 to assist the implantation of the apparatus 100 to the desired implantation position, such as shown in FIG. 12.

To ensure proper blood flow, a corresponding plug 266 can be inserted into the opening at the proximal end 104 to prevent flow of blood from within the heart 200 to a position external to the heart. After the plug 266 has been inserted into the proximal end 104 of the tubular body 106, a sheet 270 of a corresponding biocompatible material can be applied over the plug 266 and the outflow end 18 and attached over the remaining portion of the apex. For instance, the sheet can be sutured to the annular ring 210 previously applied to the apex 206. The sheet 270 of biocompatible material, for example can be formed of a chemically treated and substantially detoxified patch of tissue material, such as the NO-REACT tissue product described herein. The sheet 270 can be attached via sutures as well as other means for attaching such sheets to the myocardial tissue.

As shown in FIG. 12, the plurality of apertures 136 operate as inlets within the ventricle 208 to receive blood that flows from the patient's left atrium 264 into the left ventricle 208. The valve 116 is arranged in the apparatus thus to provide for unidirectional flow of blood from within the lumen of the tubular sidewall 106 (located in the ventricle 208) and through the outflow opening 110 residing in the aorta 224. Thus, as the ventricular pressure exceeds the aortic pressure, blood will flood from the ventricle 208, through the apparatus and into the aorta 224. As described herein, the proximal tubular portion 140 located within apex 206 of the heart 200 can permit axial lengthening and shortening of the apparatus as well as deflection transverse to the central axis of the apparatus as the heart contracts and relaxes (e.g., systolic and diastolic functions). Additionally, the supports 130 in the tubular body portion 106 between the valve 116 and the proximal portion help maintain a substantially cylindrical configuration so that the blood properly flows into the apertures 136. As described herein, additional shock absorbing structures can also be utilized to reduce the stress and strain on the apparatus as the heart continues beating.

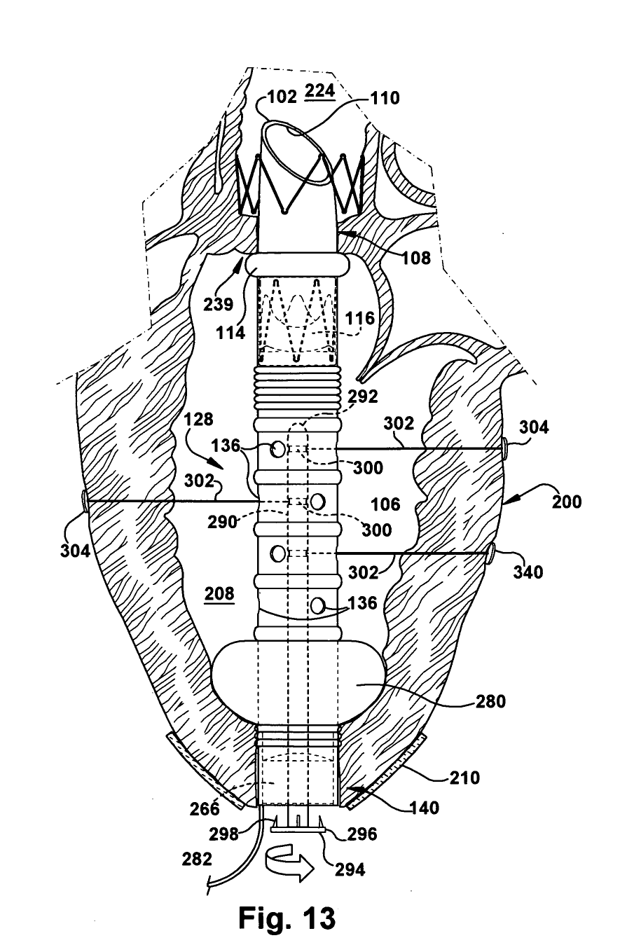

FIG. 13 depicts an example of another part of the procedure that can be utilized to decrease the volume of blood within the ventricle 208. By decreasing the volume of blood within the ventricle 208, such as in circumstances where the ventricle is dilated, the heart can remodel itself for more efficient operation. In FIG. 13, the apparatus 100 can be modified or additional structure can be added for reducing the available volume in the ventricle 208.

One approach to reduce the volume, for example, is to increase the size of the plug 266 that is inserted into the proximal end of the apparatus 100. A larger plug or other structure (which may have a fixed or variable volume) residing within the tubular sidewall 106 will displace a corresponding volume of blood, such that the volume of blood within the ventricle 208 will decrease.

Another approach is to utilize a structure external to the apparatus 100 that can be located within the ventricle to occupy a desire volume. The desired volume can be fixed or variable. For example, an adjustable or inflatable bladder 280 can be provided near the proximal end 104 of the apparatus (similar to a balloon catheter), such as surrounding the tube 106 between the proximal end portion 140 and the inflow section 128 of the tube 106. The inflatable bladder 280 thus can be inflated and deflated by introducing and withdrawing a suitable inflation fluid (e.g., saline, blood, etc.). For instance, a conduit (having an interior lumen) 282 that is in fluid communication with an interior volume of the bladder 280 can be accessible from external to the heart 200. A source of the inflation fluid (not shown) thus can be coupled to the conduit 282 for selectively introducing and withdrawing fluid from the bladder 280 so that the bladder occupies a desired volume within the ventricle 208. The bladder 280 and/or the conduit 282 can be formed as part of the apparatus 200 or each can be implemented as separate structures.

As another example, the apparatus 100 can also include an elongated rod 290 that extends axially from within the tubular portion 106 of the apparatus. For instance, the rod 290 can extend from a location adjacent the proximal end 104 and terminate in a distal end 292 at a location spaced axially apart from the valve 116. That is, the end 292 of the rod 290 is spaced axially apart from the valve 116 so as to not interfere with or contact the valve. The rod 290 can be rotatably mounted relative to the apparatus 100 so as to permits its rotation about the axis along which it extends.

For example, after the apparatus has been positioned in the ventricle 208 (e.g., as described with respect to FIGS. 11 and 12), the rod 290 can be inserted through the plug 266 along a central axis of the tubular sidewall portion 106 and be mounted loosely within the plug 54 sufficient to afford rotatably about the axis. The proximal end 294 of the rod 290 can include lateral or radial extending portion or a cylindrical portion 296 that can be gripped and, in turn rotated, about the central axis to cause corresponding rotation of the rod. One or more spikes 298 can also extend axially from the rod towards the end 104 of the apparatus 100 that can be inserted into and engage the plug 266 to restrict further rotation of the rod after the spikes or protruding portions have been inserted into the plug. Those skilled in the art will understand and appreciate various configurations and ways that the rod 290 can be restricted from axial rotation.

At least a portion of the rod 290 can extend axially through the interior of the tube 106 so as to be directly accessible through one or more of the respective apertures 136. Corresponding sutures (or other connecting elements or cords) 302 can be attached to the rod 290 and extend through the heart muscle 200 to terminate in a location that is anchored relative to the heart. For example, the sutures 302 can be anchored to the heart 200 by pledgets 304 that are applied external to the heart and tied to the respective sutures 302. The pledgets 304 can be formed of a small sheet (or plural sheets) of biocompatible material (e.g., natural tissue or synthetic or a combination of natural tissue and synthetic) that are capable of fixing an end of the suture relative to the exterior of the heart 200. Those skilled in the art will appreciate various approaches (e.g., using guide wires, long needles and the like) that can be employed to apply the sutures (or other connecting elements) 302 between the pledgets 304 and the rod 290.

By fixing the ends of the sutures 302 between the pledgets 304 and the rod 290, as the rod is rotated, the sutures can wind up onto the rod thereby shortening the length of the suture that extends between the rod and the pledgets. The shortening of the suture length between the rod 290 and the pledgets 304 operates to decrease the interior volume of the ventricle 208. As mentioned above, the reduction in ventricular volume helps remodel the heart 200, such that the heart can pump blood through the apparatus and into the aorta more efficiently. In view of the foregoing, it will be understood that the plug 266, the apparatus 100, the bladder 280 as well as the rod 290 and sutures 302 can individually, as well as in any combination thereof, provide means for reducing the interior volume of the ventricle 208.

After an appropriate reduction in the ventricular dilation has been achieved, the rod 290 can be anchored into the plug 266 or otherwise fixed relative to the heart 200 to maintain relatively substantially fixed rotational position. It is to be understood and appreciated that subsequent adjustments can be made to reduce further dilation at a subsequent time. For example after a period of time (e.g., days or weeks or months) after the heart has remodeled itself to the new reduced dilation configuration, the elongated rod 290 can be rotated further to cause further reduction in dilation until a desired size of the ventricle has been achieved. At some point, if desired, the rod 290 and sutures 302 can be removed from the heart 200, such as by disconnecting the pledgets 304 and retracting the rod and attached sutures back through the plug 266.

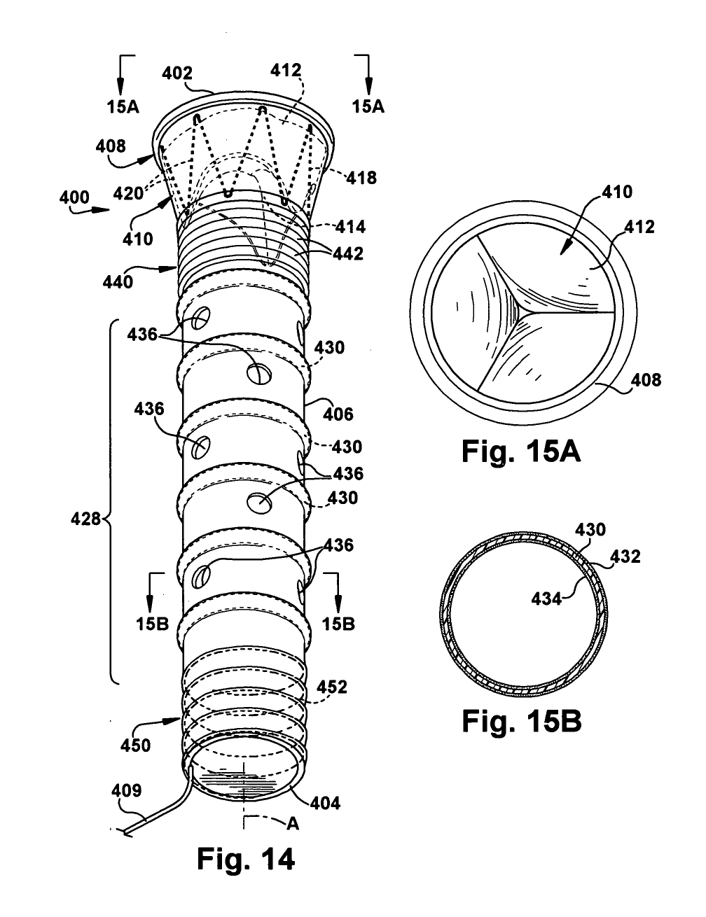

FIGS. 14 and 15 depict an example of another embodiment of an apparatus 400 that can be implanted within a patient's ventricle. The example of FIG. 14 provides an arrangement configured for providing for substantially unidirectional flow of blood from a patient's left atrium into the patient's left ventricle. That is, the apparatus 400 can replace the function of the patient's mitral valve (e.g., the patient's native valve or another prosthetic valve). The apparatus 400 has a substantially tubular configuration that extends between respective ends 402 and 404 that are spaced apart by an associated sidewall 406. In the example of FIG. 14, the end 402 corresponds to an inflow end that can be positioned at the mitral valve annulus. The apparatus 400 can be substantially straight or it can be curved along its central axis A extending through the apparatus.

The apparatus 400 also includes a valve portion 408 at the inflow end. At least a portion of valve portion has a greater exterior cross-sectional diameter than the length of the sidewall 406 that extending from the outflow end of the valve portion. In the example embodiment of FIG. 14, the valve portion is depicted as having a substantially conical frustum configuration in which the inflow end has a greater diameter than the outflow end and the sidewall between such ends tapers to approximate the diameter of the sidewall 406. Other shapes can also be utilized for the valve portion including, for example, cylindrical or axially curved sidewall.

The valve portion 408 includes a valve 410 positioned to provide for substantially unidirectional flow of blood into an interior volume of the apparatus 400 defined by the sidewall 406. The valve 410 can be mounted within a length of the tubular sidewall. Alternatively, the valve 410 can be attached at an end of the tubular sidewall 406 (e.g., end-to-end anastomosis). The valve 410 can include an inflow end 412 located adjacent the inflow end 402. An outflow end 414 of the valve 410 can be spaced axially apart from the inflow end 412, which spacing can vary according to the type and configuration of valve.

By way of example, the valve 410 can be a natural tissue heart valve prosthesis, such as may be shown and described in the above-incorporated patent application. While the valve 410 is shown and described as a natural tissue valve prosthesis, those skilled in the art will understand and appreciate that other types of valves can also be utilized in the apparatus 400. For instance, other types and configurations of natural tissue valve prosthesis as well as mechanical valves or biomechanical valves or any combination or hybrid of these types of valves can also be utilized.

The valve 410 can also include a support structure 418 that helps maintain the valve in a desired configuration to provide for proper coaptation of the valve leaflets to provide for substantially unidirectional flow of blood through the valve. In the depicted embodiment, the support structure 418 has a greater cross-sectional dimension (e.g., diameter) at the inflow end 412 than at the outflow end 414, such as corresponding to a frusto-conical configuration. In the example of FIG. 14, the support structure 418 includes a plurality of generally axially extending support features 420 that extend between the ends 412 and 414 of the valve. Adjacent pairs of the support features 420 are interconnected at the respective ends 412 and 414 at an angular junction. The interconnection between adjacent support features 420 further can be biased (e.g., corresponding to a spring or other means) to expand radially outwardly relative to the central axis extending through the valve.

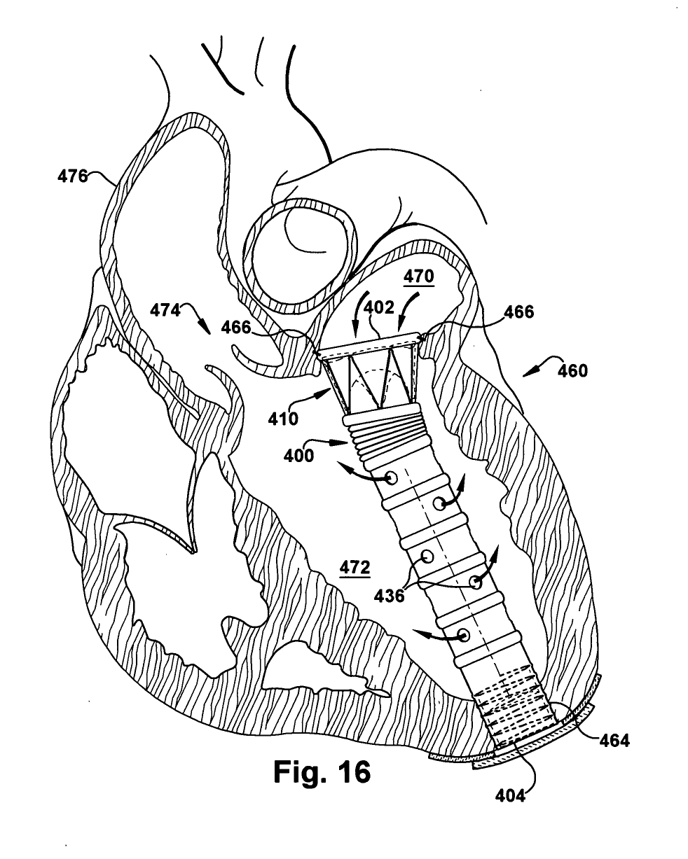

The support structure 418 can be self-expanding or otherwise be expandable by mechanical or other means. For example, one or more sutures (or other retaining structure) can be applied around the exterior of the support 418 structure to hold it against the exterior sidewall of the valve 410. Alternatively, a tubular sheet of a corresponding biocompatible material can be applied around the structure to hold in its desired compressed orientation. In its compressed condition, insertion of the apparatus 400 into a patient's heart valve (e.g., a mitral valve can be facilitated. Once in an appropriate position, the retaining structure (e.g., suture and/or tube of tissue) can be removed and the support structure 418 can be expanded into engagement with surrounding tissue (e.g., at the mitral position—see FIG. 16).

The portion of the sidewall 406 extending between the valve 410 and the end 404 defines an outflow section 428 of the apparatus 400. The outflow section 428 can be supported by a plurality of support members 430. In the example of FIG. 14, the support members 430 are illustrated as annular structures that are spaced axially apart from each other along the length of the inflow section 428 (e.g., short cylindrical rings). The support members 430 can be mounted between corresponding tubular sheets 432 and 434 of a biocompatible flexible material, such as described herein. Similar to as discussed with respect to other embodiments described herein, an animal tissue material having been treated and substantially detoxified (e.g., a NO-REACT tissue product) can be utilized to provide a covering for the inner and outer surfaces of the apparatus 400. For example, the sheets 432 and 434 can be formed of such animal tissue material.

A plurality of apertures 436 can be formed through the sidewall 406 of the outflow section 428, including through both of the sheets 432 and 434. The apertures 436 can be at axial spaced apart locations between adjacent pairs of the respective support members 430. In this way, the softer more compliant sheets of flexible material that extend between the apertures 436 can be supported by the respective support members to maintain a desired configuration of the outflow section 428 of the sidewall 406. As one example, the aggregate area of the apertures 436 can at least approximate the cross-sectional area of the inflow end of the valve 410 (e.g., totaling about 5 cm² given a 2.5 cm diameter valve). In this way, the flow of blood through the valve 410 and into the interior volume of the outflow section and through the apertures 436 can be facilitated.

An elongated portion 440 of the tubular sidewall 406 between the outflow section 428 and the valve 410 can be configured to provide for axial elongation, axial compression and radial (or angular) deflection relative to the central axis. In this regard, the portion 440 can include one or more springs (e.g., helical springs or windings) 442 encapsulated within a biocompatible material. For instance, the springs 442 can be mounted between the sidewall sheets 432 and 434 of the biocompatible material. The same biocompatible materials further may form the interior and exterior surfaces of the other parts of the sidewall 406. Additionally, to facilitate elongation compression and transverse movement of the portion 440 relative to the central axis, the sidewall (that is formed by the interior and exterior tubular sheets 432 and 434 of biocompatible material) may be corrugated or otherwise configured similar to a bellow (e.g., similar to an accordion) to permit desired movement thereof. Those skilled in the art will understand and appreciate various types and configurations of materials that can provide for suitable movement and sufficient support at the proximal end portion 440.

A similarly configured elongated portion 450 can also be provided adjacent the end 404, such as between the outflow portion 428 and the end 404. The elongated portion thus can be configured to provide for axial elongation, axial compression and radial (or angular) deflection relative to the central axis. The portion 450 can be corrugated as well as include one or more springs 452 that help maintain the desired tubular configuration while also permitting desired flexion and movement of the elongated portion.

FIG. 16 depicts an example in which the apparatus 400 of FIG. 14 has been implanted in a patient's heart 460 extending between the mitral valve annulus 462 and the apex 464 of the patient's heart. The apparatus 400 can provide a useful technique to replace the function of the patient's mitral valve, such as when the mitral valve exhibits stenosis or is otherwise insufficient. The apparatus 400 can be implanted within the patient's valve or it can be implanted after a portion of the valve has been removed. The implantation procedure can be substantially similar to that shown and described above with respect to FIGS. 7-12. Thus, the apparatus 400 can be implanted during a minimally invasive procedure that does not require cardiopulmonary bypass.

Briefly stated, an opening can be formed in the apex 464, such as by an appropriate cutting tool. The opening can be enlarged (e.g., by a trocar or other instrument) to provide an opening sufficiently large to accommodate the end portion 450 of the apparatus 400. If the mitral valve is calcified, a portion of the calcified valve can be removed by a procedure similar to that shown and described with respect to FIG. 9. For instance, increasingly larger cutting instruments can be utilized until the desired portions of the patient's mitral valve have been excised. Alternatively, the apparatus 400 can be implanted within the patient's mitral valve, without first removing the patient's mitral valve. That is, the inflow end 402 of the apparatus 400 can be implanted within the patient's mitral valve. For example, as mentioned above with respect to FIG. 14, the valve portion 408 can be expanded (e.g., either automatically or by manual means, such as balloon catheter or other expansion tool) so that an exterior of the valve portion has an increased diameter that conforms to the patient's mitral annulus.

After the mitral valve has been prepared (as may be needed), the apparatus 400 can be inserted through the opening in the apex and into its implantation position, such as depicted in FIG. 16. In its implantation position, the protruding portion 408 can be positioned at the mitral position, such as several millimeters (e.g., from about 5 mm to about 10 mm) into the atrium 470. The sidewall portion 406 extends from the protruding portion to the end 404 that is secured at the apex 464 of the patient's heart. One or more sutures 466 can be applied externally to secure the inflow end 402 of the apparatus relative to mitral valve annulus of the patient's heart. For example, the sutures 466 can be applied through the heart and into the protruding portion 408 or other structure of the apparatus near the inflow end 402.

In the implanted position, the outflow portion 428 of the apparatus resides in the patient's left ventricle 472, extending between the apex 464 and the mitral valve annulus 462. Thus, the apparatus provides for blood flow from the left atrium 470 through the valve 410, into the lumen defined by the outflow portion 428, through the apertures 436 and into the patient's ventricle 472. From the left ventricle 472, blood can flood through the patient's aortic valve 474 and into the patient's aorta 476 in a normal manner. As described herein, additional shock absorbing structures can also be utilized to reduce the stress and strain on the apparatus 400 as the heart continues beating.

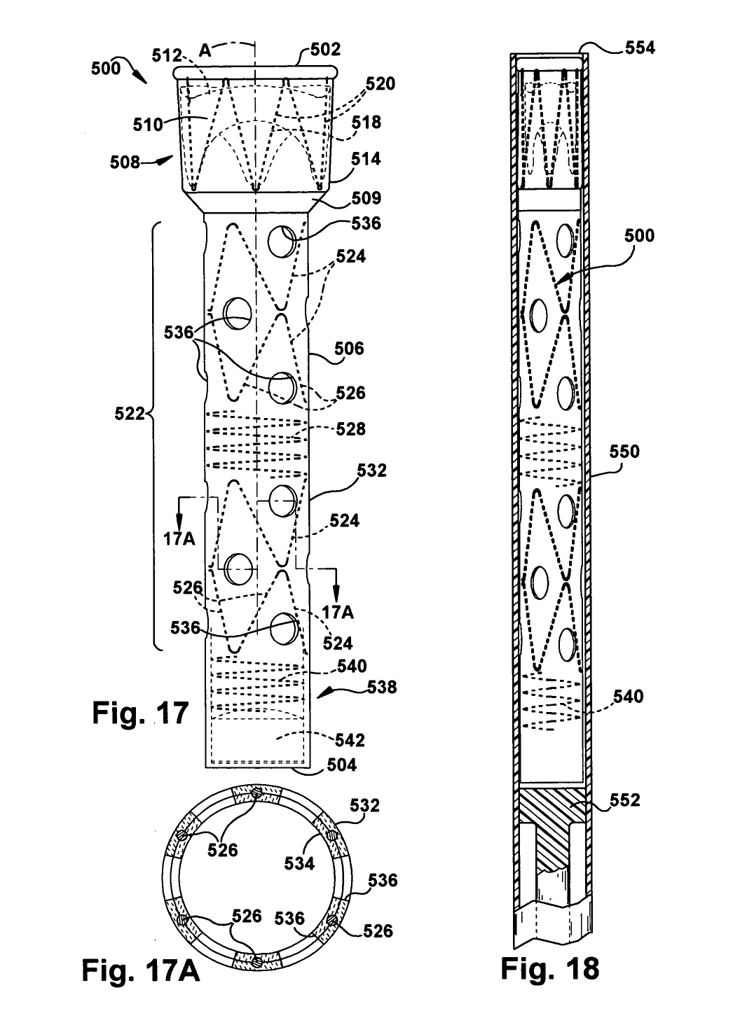

FIG. 17 depicts an example of another embodiment of an apparatus 500 that can be implanted within a patient's ventricle for providing for substantially unidirectional flow of blood from a patient's left atrium into the patient's left ventricle. That is, the apparatus 500 can replace the function of the patient's mitral valve (e.g., the patient's native valve or another prosthetic valve). The apparatus 500 has a substantially tubular configuration that extends between an inflow end 502 and an outflow end 504, which ends are spaced apart from each other a generally cylindrical sidewall 506. The apparatus 500 can be substantially straight or it can be curved along its central axis A extending through the apparatus.

The apparatus 500 also includes a valve portion 508 at the inflow end. In the example of FIG. 17, the valve portion 508 has a substantially configuration and a diameter that is greater (e.g., by about 20% greater) than the diameter of the adjacent length of the sidewall that extends from the valve portion. A frusto-conical portion 509 can interconnect the two different diameter portions 508 and 506 of the apparatus 500. The portion 509 can be supported (e.g., by a spring or other support structures) or unsupported (formed of one or more sheets of flexible material) to provide the desired configuration to merge the valve portion with the rest of the sidewall. Other shapes can also be utilized for the valve portion, such as shown and described herein.

The valve portion 508 includes a valve 510 configured to provide for substantially unidirectional flow of blood into an interior volume of the apparatus 500 defined by the sidewall 506. The valve 510 can be mounted within a length of the tubular sidewall. Alternatively, the valve 510 can be attached at an end of the tubular sidewall 506 (e.g., end-to-end anastomosis). The valve 510 can include an inflow end 512 located adjacent the inflow end 502. An outflow end 514 of the valve 510 can be spaced axially apart from the inflow end 512, which spacing can vary according to the type and configuration of valve.

By way of example, the valve 510 can be a natural tissue heart valve prosthesis, such as may be shown and described in the above-incorporated patent application. Those skilled in the art will appreciate that other embodiments can include other types and configurations of natural tissue valve prosthesis or include mechanical valves or biomechanical valves or any combination or hybrid of these types of valves can also be utilized.

The valve 510 can also include a support structure 518 that helps maintain the valve in a desired dimension and configuration. For the example of a natural tissue valve, the support structure 518 helps to provide for proper coaptation of the valve leaflets to provide for substantially unidirectional flow of blood through the valve. In the depicted embodiment, the support structure 518 has a substantially cylindrical cross-sectional configuration and a diameter that is greater than the diameter of the adjacent portion of the sidewall 506 that extends from the valve portion 508. The support structure 518 includes a plurality of generally axially extending support features 520 that extend between the ends 512 and 514 of the valve. Adjacent pairs of the support features 520 are interconnected at the respective ends 512 and 514 at an angular junction. The interconnection between adjacent support features 520 further can be biased (e.g., corresponding to a spring or other means) to urge adjacent pairs of the axially extending support features apart and, thereby, provide for radial outward expansion expand of the support 518 relative to the central axis extending through the valve portion 508. The support structure 518 can be self-expanding or otherwise be expandable by mechanical or other means.

The portion of the sidewall 506 extending between the valve 510 and the end 504 defines an outflow section 522 of the apparatus 500. The outflow section 522 can be supported by a plurality of support members 524. In the example of FIG. 17, the support members 524 are illustrated as substantially sinusoidal annular structures similar to the support 518 of the valve 510. For instance, each of the support members 524 includes a plurality of axially extending and interconnected features 526 that extend between axially spaced apart ends of each support member along a zig-zag or sinusoidal path. Each axially adjacent pair of support members 524 can be attached to each other at the respective ends (e.g., by sutures, welding, adhesive, or the like) to provide the elongated support structure. Additionally or alternatively, one or more support spring 528 can be provided between and axially spaced apart a pair of axially adjacent support member 524. The spring 528 affords additional flexibility to provide for axial compression and elongation as well as can permit additional radial deflection of the elongated sidewall 506.

The support members 524 and the spring 528 can be mounted between corresponding tubular sheets 532 and 534 of a biocompatible flexible material, such as described herein (See FIG. 17A). Similar to as discussed with respect to other embodiments described herein, an animal tissue material having been treated and substantially detoxified (e.g., a NO-REACT tissue product) can be utilized to provide a covering for the inner and outer surfaces of the apparatus 500. For example, the sheets 532 and 534 can be formed of such animal tissue material. Other flexible biocompatible natural tissue as well as synthetic materials can also be utilized.

A plurality of apertures 536 can be formed through the sidewall 506 of the outflow section 528, including through both of the sheets 532 and 534. The apertures 536 can be at axial spaced apart locations between adjacent pairs of the respective support features 526. In this way, the softer and more compliant sheets of the flexible material that extend over the support features 526 can operate to maintain the apertures 536 open as well as maintain a desired configuration of the outflow section 528 of the sidewall 506. As one example, the aggregate area of the apertures 536 can at least approximate the cross-sectional area of the inflow end of the valve 510 (e.g., totaling about 5 cm² given a 2.5 cm diameter valve). In this way, the flow of blood through the valve 510 and into the interior volume of the outflow section can be accommodated through the apertures 536.

The prosthesis can include a proximal end portion 538 can also be provided adjacent the end 504. The end portion 538 may include one or more springs 540 that help maintain the desired tubular configuration while also permitting desired flexion and movement of the elongated portion after it is implanted in the patient's heart. The spring 540 can be sandwiched between the sheets 532 and 534 similar to the spring 528 and the other support structures 524. Additionally, when the prosthesis is implanted, a plug 542 can be inserted into the end 504 to close the end. Alternatively, the end 504 can be closed.

As shown in FIG. 18, the prosthesis 500 can be compressed into a desired compressed and reduced cross-sectional configuration to assist with implantation. For example, the apparatus 500 can be inserted within an elongated tubular structure (e.g., an implanter such as a trocar) 550 having a diameter that is less than the normal diameter of the valve portion 508. The diameter of the tubular structure 550 can also be less than the normal diameter of the sidewall 506.

By way of further example, the supports 518, 524 and 528 can be formed of a material and be configured to permit radial compression and expansion back to a normal, desired dimensions and configuration. As one example, the supports 518, 524 and 528 can be formed of a shape memory alloy, such as nitinol (nickel-titanium alloy). For instance, the apparatus 500 may be cooled, such as by being introduced to a cooling solution (e.g., water), and then compressed to facilitate its insertion into an interior of the tubular structure 550. The supports 518, 524 and 528 can then be bent or deformed to a reduced cross-sectional dimension when in its low-temperature (martensitic) form to facilitate its mounting within the tubular structure 550, such part of an implanter. When the supports 518, 524 and 528 are heated to its transformation temperature, which may vary according to the alloy composition, it quickly reverts to its high-temperature (austenitic) form. The apparatus 500 may retain the compressed condition by keeping it cooled. Alternatively, the apparatus 500 may be retained in the compressed position, such as with sutures circumscribing the structure, a cylindrical enclosure around the structure, etc. With the tubular structure being part of an implanter, it includes a plunger (or other structure) 552 that is moveable within the interior to discharge the apparatus 500 from an open end 554 thereof.

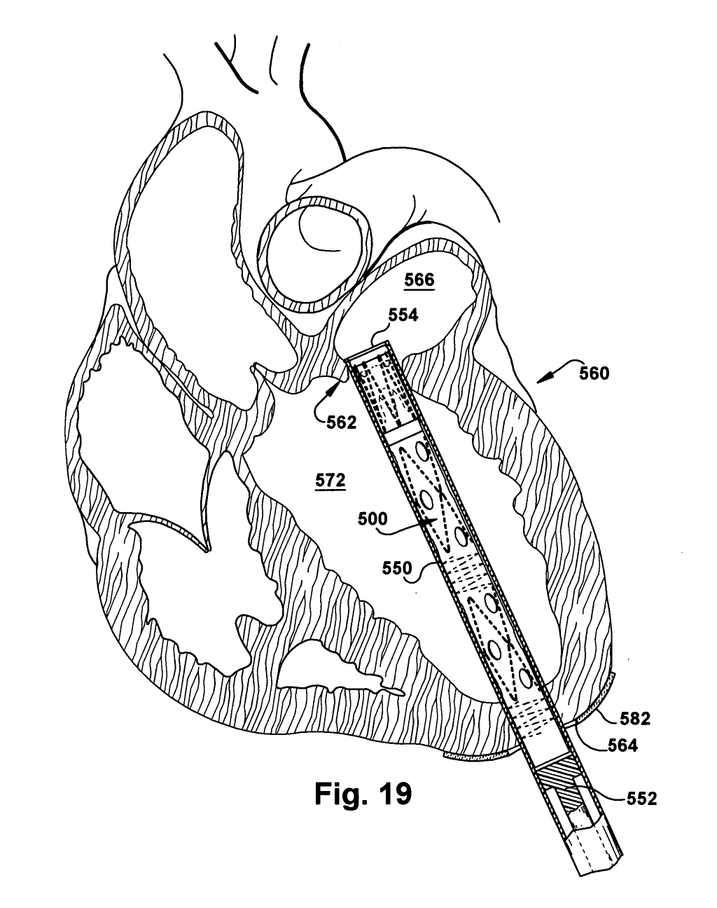

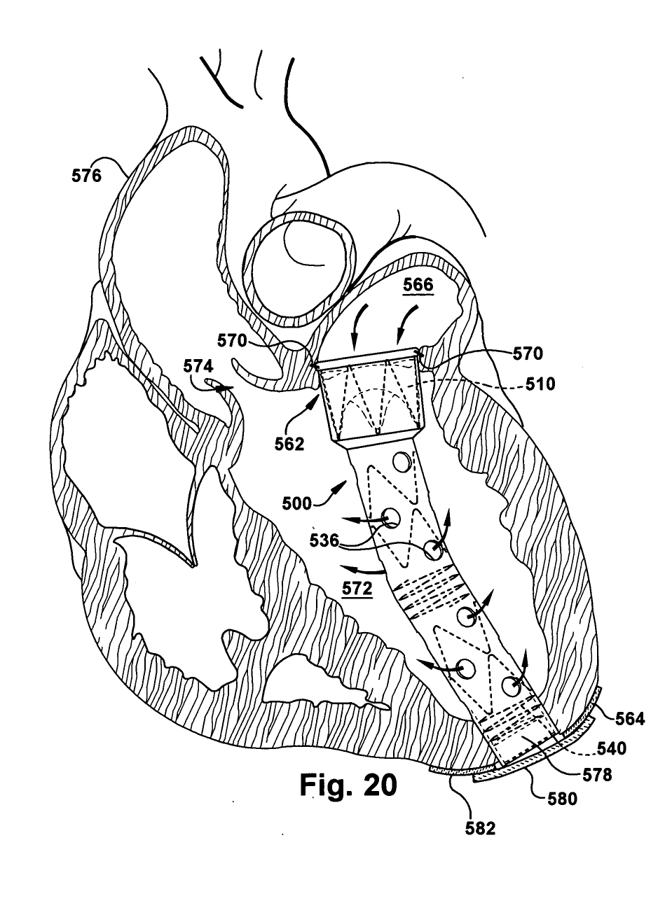

FIGS. 19 and 20 depict parts of a procedure that can be performed to implant the apparatus 500. The implantation procedure can be substantially similar to that shown and described above with respect to FIGS. 7-12. Thus, the apparatus 500 can be implanted during a minimally invasive procedure that does not require cardio-pulmonary bypass. The apparatus 500 can be implanted within the patient's valve or it can be implanted after a portion of the valve has been excised. The implantation procedure can be substantially similar to that shown and described above with respect to FIGS. 7-12. Thus, the apparatus 500 can be implanted during a minimally invasive procedure that does not require cardio-pulmonary bypass.

After the mitral annulus 562 has been prepared (as needed), a barrel 550 of the implanter can be inserted through the apex 564 to position the open discharge end 554 at the annulus. FIG. 19 depicts an example of an implanter positioned within a heart 560 for implanting the apparatus 500 intraventricularly. For example, the end 554 can be positioned slightly (e.g., about 2-7 mm) into the left atrium 566, such as by palpating the exterior of the heart 560. Once in an appropriate position, the apparatus can be discharged by activating the plunger 552 simultaneously with the removal of the implanter from the heart 560. In this way, the inflow end 502 of the apparatus 500 can be implanted at a desired position at the mitral annulus 562. As the apparatus 500 is discharged from the implanter, the apparatus can expand to its normal dimension and configuration. The expansion can be self-expanding or it can be expanded by a balloon catheter or other means for expanding the apparatus (located internal or external to the apparatus). Those skilled in the art will understand and appreciate various types and configurations of implanters that can be utilized to carry out the implantation described above.

FIG. 20 depicts an example in which the apparatus 500 of FIG. 17 has been implanted in a patient's heart 560. The implanted apparatus 500 extends between the mitral valve annulus 562 and the apex 564 of the patient's heart 560. The apparatus 500 thus can provide a useful technique to replace the function of the patient's mitral valve, such as when the mitral valve exhibits stenosis or is otherwise insufficient.

One or more sutures 570 can be applied externally to secure the inflow end 502 of the apparatus 500 relative to mitral valve annulus 562 of the patient's heart 560. For example, the sutures 566 can be applied through the heart and into the inner and outer sheets of tissue 532 and 534 near the inflow end 502.

In the implanted position, the outflow portion 528 of the apparatus resides in the patient's left ventricle 572, extending between the apex 564 and the mitral valve annulus 562. Thus, the apparatus provides for blood flow from the left atrium 566 through the valve 510, into the lumen defined by the outflow portion 528, through the apertures 536 and into the patient's ventricle 572. From the left ventricle 572, blood can flood through the patient's aortic valve 574 and into the patient's aorta 576 in a normal manner.

To ensure proper blood flow, a corresponding plug 578 can be inserted into the opening at the proximal end 504 to prevent flow of blood from within the heart 200 to a position external to the heart. After the plug 578 has been inserted into and secured relative to the proximal end 504, a sheet 580 of a corresponding biocompatible material can be applied over the plug 578 and the outflow end 504 and attached over the a portion of the apex 564. For instance, the sheet 580 can be sutured to an annular ring 582 previously applied to the apex 564, such as prior to or commensurate with forming the aperture in the apex to provide access to the ventricle 572. The sheet 580 of biocompatible material, for example can be formed of a chemically treated and substantially detoxified patch of tissue material, such as the NO-REACT tissue product described herein. The sheet 582 can be attached via sutures as well as other known means for attaching such sheets to the myocardial tissue.

It will be understood that the various features shown and described with respect to the apparatuses for aortic valve replacement and mitral valve replacement can be utilized interchangeably in each of the respective apparatus shown and described herein. As one example, the support structures utilized in the apparatus of FIG. 17 can be used in an intraventricular apparatus to replace the function of a patient's aortic valve. Additionally, while the example embodiments shown and described herein relate to use of a single apparatus in the patient's heart, it is contemplated that more than one such apparatus can be implanted to replace valvular function of more than one valve (e.g., aortic and mitral valves).

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. An intraventricular apparatus, comprising:
a substantially tubular sidewall that extends substantially axially between spaced apart first and second ends, an opening located proximal the first end;
a valve located adjacent the first end to provide for substantially unidirectional flow of blood therethrough; and
at least one aperture extending through the tubular sidewall at an axial location between the valve and the second end to provide for substantially free flow of blood between an interior and an exterior of the sidewall.

2. The apparatus of claim 4, further comprising a protruding member that extends radially outwardly from an exterior of the tubular sidewall at an axial location between the valve and the outflow opening.

3. The apparatus of claim 2, wherein the protruding member comprises an inflatable member having a variable interior volume that defines the radial distance that the protruding member extends relative to the exterior of the tubular sidewall.

4. The apparatus of claim 1, further comprising a plug mountable at an opening located at the second end to close the opening located at the second end.

5. The apparatus of claim 1, wherein the at least one aperture comprises a plurality of spaced apart apertures that extend through the tubular sidewall at respective axial locations between the valve and the second end, such that fluid can flow through the apertures and through the valve.

6. The apparatus of claim 1, wherein the tubular sidewall further comprises an interior sidewall surface and an exterior sidewall surface of a biocompatible material.

7. The apparatus of claim 6, wherein the biocompatible material comprises a biocompatible biological material.

8. The apparatus of claim 7, wherein the biocompatible material comprises an animal tissue material.

9. The apparatus of claim 1, wherein the valve comprises a natural tissue heart valve.

10. The apparatus of claim 1, further comprising at least one support member attached to the tubular sidewall adjacent the at least one aperture to maintain an adjacent part of the tubular sidewall in a substantially cylindrical configuration.

11. The apparatus of claim 10, wherein the at least one support member further comprises a plurality of support rings spaced axially apart from each other between the valve and the second end of the tubular sidewall.

12. The apparatus of claim 10, wherein the at least one support member further comprises at least one spring operatively connected with the tubular sidewall between the valve and the second end of the tubular sidewall.

13. The apparatus of claim 1, further comprising a tip portion of the apparatus at the first end that defines a cannula tip portion having a generally frusto-conical sidewall configuration that tapers to a smaller diameter thereof at the first end.

14. The apparatus of claim 1, wherein at least one axial length of the tubular sidewall located axially between the valve and the second end is configured to permit at least one of axial elongation, axial compression and radial deflection of the tubular sidewall relative to a central axis thereof.

15. The apparatus of claim 1, further comprising means for reducing an interior volume of a ventricle in which the apparatus is implanted.

16. An intraventricular apparatus comprising:
an elongated substantially cylindrical sidewall that extends substantially longitudinally between spaced apart first and second ends;
an opening at the first end that permits flow of fluid relative to an interior lumen of the elongated body;
a valve located between the opening and an axial length of the sidewall that extends between the valve and the second end, the valve permitting substantially unidirectional flow of fluid through the valve; and
a plurality of apertures through an inflow section of the sidewall between the valve and the second end to permit substantially free flow of blood between an interior of the inflow section and a location external to the inflow section; and
at least one support member to maintain a substantially cylindrical configuration of the sidewall near at least some of the plurality of apertures.

17. The apparatus of claim 16, further comprising a protruding member that extends radially outwardly from an exterior sidewall of the elongated body at an axial location between the valve and the opening.

18. The apparatus of claim 16, further comprising at least one sheet of a biocompatible material that provides a radially interior surface and a radially exterior surface of the apparatus.

19. The apparatus of claim 16, wherein the at least one support member further comprises a plurality of axially spaced apart support rings located between the valve and the second end of the sidewall.

20. The apparatus of claim 16, wherein the at least one support member further comprises at least one spring operatively connected with the sidewall between the valve and the second end of the sidewall.

21. The apparatus of claim 16, wherein at least one section of the sidewall located axially between the valve and the second end is configured to permit at least one of axial elongation, axial compression and radial deflection of the sidewall relative to a central axis extending through the sidewall.

22. An intraventricular apparatus comprising:
elongated means for providing a lumen that extends within a substantially cylindrical sidewall between spaced apart first and second ends;
means for providing an opening at the first end of the elongated means that permits flow of fluid through the opening relative to an interior of the elongated means;
means for providing substantially unidirectional flow of fluid axially through a portion of the elongated means proximal the first end of the elongated means;
means for permitting substantially free flow of the fluid through the sidewall of the elongated means; and
means for maintaining a substantially cylindrical configuration of the sidewall of the elongated means between the second end and the means for providing unidirectional flow of fluid.
